(19) United States
(12) Patent Application Publication
(10) Pub. No.: US 2022/0202978 A1
(43) Pub. Date: Jun. 30, 2022

(54) Disinfecting Diffuser Device

(71) Applicant: AMRICH INC. LIMITED, Alberta (CA)

(72) Inventors: GuangXu Zhu, Ottawa (CA); Tanveer Ahmad, Ottawa (CA)

(73) Assignee: AMRICH INC. LIMITED, Alberta (CA)

(21) Appl. No.: 17/560,066

(22) Filed: Dec. 22, 2021

Related U.S. Application Data
(60) Provisional application No. 63/129,831, filed on Dec. 23, 2020.

(51) Int. Cl. A61L 2/22; A61L 2/26; A61L 9/14

(52) U.S. Cl. CPC A61L 2/22; A61L 2101/50; A61L 9/14; A61L 2/26

(57) ABSTRACT

A disinfecting device for purifying air to reduce the presence of dust and/or pathogens. The device includes a main housing, a battery module configured for installation within a first receptacle in the main housing and a reservoir module configured for installation within a second receptacle in the main housing. The reservoir is configured to hold a disinfecting fluid. The housing also includes an ultrasonic wave emitter electrical power communication with the battery, the ultrasonic wave emitter providing ultrasonic waves to the disinfecting fluid thereby creating a pressurized fluid, a membrane in contact with the pressurized fluid. The membrane generates reduced-size particles of the pressurized fluid which are emitted from a nozzle mounted on the housing.

### CROSS-REFERENCES TO RELATED APPLICATIONS

This application claims priority from U.S. Provisional Application Ser. No. 63/129,831 filed Dec. 23, 2020, which is hereby incorporated herein by reference in its entirety.

### FIELD

The technology relates to release of disinfecting agents into the air to reduce the presence of dust, pathogens and other particulates, and more particularly to a device configured to emit a disinfecting agent by aerosol diffusion.

### BACKGROUND

Bacterial, viral and fungal contamination of rooms and other confined spaces, such as air conditioning duct-work, has long been a source of infection for humans, particularly in dwelling areas, hospitals, classrooms, and public restrooms. Bacteria, viruses, and other pathogens adhere to surfaces after contact with humans and also linger in the air within a room after being discharged by a person sneezing or coughing. In this way, humans spread pathogens such as rhinoviruses, coronaviruses, influenza viruses, rotaviruses, hepatitis viruses, tuberculosis, and conjunctivitis-causing viruses as well as various strains of bacteria causing staphylococcal and streptococcal bacterial infections, among others.

Chitosan is a linear polysaccharide composed of randomly distributed β-(1→4)-linked D-glucosamine (deacetylated unit) and N-acetyl-D-glucosamine (acetylated unit). It is made by treating the chitin shells of shrimp and other crustaceans with an alkaline substance, such as sodium hydroxide. Chitosan and derivatives have been explored in the development of nanomaterials, bioadhesives, wound dressing materials, improved drug delivery systems, enteric coatings, and as antifungal, antibacterial and antiviral compositions.

Devices for dispensing aerosolized or pressurized fluids from a container have been described. One such device described in U.S. Pat. No. 3,137,414, incorporated herein by reference in its entirety, proposes an aerosol can assembly and an activating cap that can be set into an operating position so that the contents of the container can be completely dispensed therefrom. U.S. Pat. No. 3,325,064, incorporated herein by reference in its entirety, describes a device designed to depress an aerosol actuator until the contents of the container are completely discharged. U.S. Pat. No. 4,882,873, incorporated herein by reference in its entirety, describes using these aerosol containers as total release bombs for effectively fogging a room to eradicate insect infestations.

There continues to be a need for development of improved devices for disinfecting indoor spaces.

### SUMMARY

In accordance with one aspect of the technology, a disinfecting device is provided. The device includes a main housing and a battery module configured for installation within a first receptacle in the main housing. The reservoir module is configured for installation within a second receptacle in the main housing. The reservoir configured to hold a disinfecting fluid. The device includes an ultrasonic wave emitter mounted in the housing and in electrical power communication with the battery such that the ultrasonic wave emitter provides ultrasonic waves to the disinfecting fluid thereby creating a pressurized fluid. There is a membrane in contact with the pressurized fluid, for generating reduced-size particles of the pressurized fluid. A nozzle is mounted on the main housing for receiving and emitting a stream of the reduced-size particles of disinfecting fluid.

The reservoir may include a temperature controller in electrical power communication with the battery. The device may further include a user interface screen configured to display an indicator selected from the group consisting of: power status, battery level and reservoir fluid level.

In some embodiments, the housing includes a conduit extending from the reservoir to the membrane. The ultrasonic wave emitter is configured to emit ultrasonic waves to the disinfecting fluid in the conduit.

In some embodiments, the nozzle is configured to generate the reduced-size particles in an electrostatic spray. In some embodiments, the nozzle has an inner cavity coated with a film of chitosan fibers. The chitosan fibers may be negatively charged.

In some embodiments, the membrane is a chitosan nanofiber membrane. The chitosan nanofiber membrane may have openings with a mean diameter in a range of about 500 nm to about 2.2 μm

In some embodiments, the reservoir module is removable and includes a port for refilling the reservoir. The reservoir module may be configured to hold a volume of about 7 mL to about 15 mL.

In some embodiments, operation of the device is controlled by a housing cover in slidable contact with an upper surface of the housing, the housing cover inactivating the electrical power in a first position on the housing which covers the nozzle, and activating the electrical power in a second position on the housing which exposes the nozzle. The screen may be mounted on the housing cover.

In some embodiments, the ultrasonic wave emitter generates ultrasonic waves in a range of about 1.6 mHz to about 1.8 mHz.

Another aspect of the technology is a disinfecting kit which includes any embodiment of the device described herein and instructions for operating the disinfecting device. The kit may also include a disinfecting fluid, which may be a chitosan solution. The chitosan solution may include a derivative of chitosan including cross-linkages and/or at least one chemical modification. The chemical modification may be a C-3 O-sulfonyl modification. The kit may also include a battery charger compatible with the battery module.

### BRIEF DESCRIPTION OF THE DRAWINGS

Various objects, features and advantages of the technology will be apparent from the following description of particular embodiments of the technology, as illustrated in the accompanying drawings. The drawings are not necessarily to scale. Instead, emphasis is placed upon illustrating the principles of various embodiments of the technology.

FIG. 1A is an illustration of one embodiment of a personal disinfecting diffuser device 10 in the operational state emitting an atomized disinfecting fluid from a nozzle.

FIG. 1B is an illustration of the personal disinfecting diffuser device 10 of FIG. 1A in the shut-off state, wherein the cover 14 is disposed over the nozzle 18.

FIG. 2A is a side elevation view of the diffuser device 10 in the operational state as shown in FIG. 1A.

FIG. 2B is a side elevation view of the diffuser device 10 in the shut-off state as shown in FIG. 1A.

FIG. 3 is a schematic view illustrating functional components of the diffuser device 10 with the cover shifted to the left side of the device 10.

### DETAILED DESCRIPTION

Most disinfectants are sold as liquid compositions that can be sprayed on surfaces to kill bacteria, viruses, and other microbes. However, for a person to spray an entire room with disinfectant using the current consumer-sized spray cans would be both tedious and time-consuming. Currently, no known disinfectants or disinfectant methods are efficient to disinfect a room effectively and in a reasonable amount of time.

The inventors of the present application have recognized that individuals are becoming increasingly concerned with air quality, particularly in light of the COVID-19 pandemic caused by SARS-CoV-2 which has been recognized as an airborne pathogen. There is an increasing desire to ensure personal safety.

In recognition that individuals would like to have more control over their personal safety, the inventors have conceived of a personal hand-held device with the capability to emit a fine mist or fog including an environmentally acceptable natural disinfecting agent based on the natural carbohydrate chitosan. The embodiment of the device described herein is configured to hold disinfecting fluid in a reservoir which also contains an ultrasonic emitter. The ultrasonic emitter generates high-frequency vibrations of the disinfecting fluid to generate reduced-size fluid particles, which may be as small as nanoscale particles. The pressure generated in the fluid as a result of the operation of the ultrasonic emitter forces the fluid particles through a chitosan fiber membrane and causes emission of fluid in the form of nano-filaments from the device as a mist. The nano-filament mist purifies the air in the environment of the user by absorbing dust, bacteria, viruses and other airborne particulates.

Various aspects of the technology will now be described with reference to an example embodiment illustrated in the figures. For the purposes of illustration, components depicted in the figures are not necessarily drawn to scale. Instead, emphasis is placed on highlighting the various contributions of the components to the functionality of various aspects of the technology. A number of possible alternative features are introduced during the course of this description. It is to be understood that, according to the knowledge and judgment of persons skilled in the art, such alternative features may be substituted in various combinations to arrive at different embodiments of the present technology.

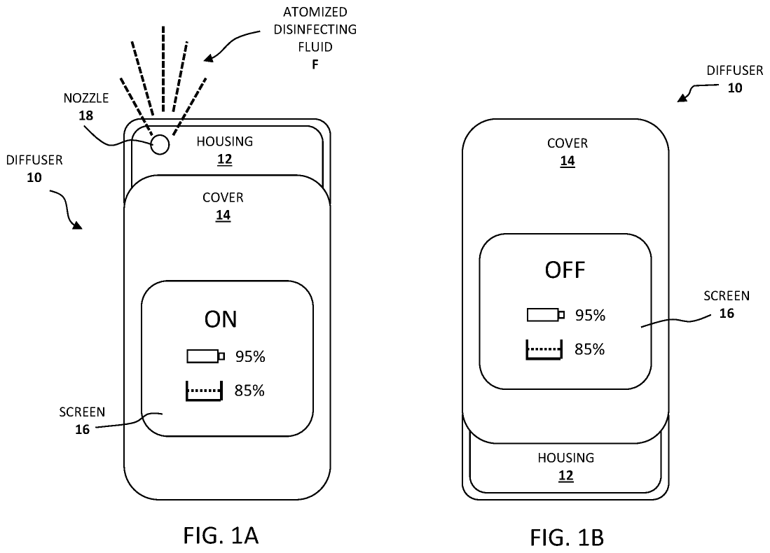

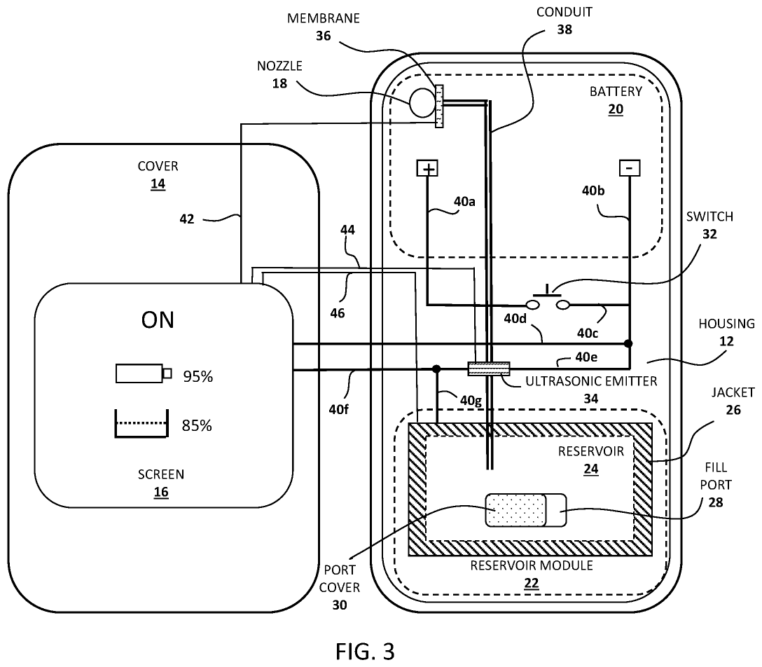

Turning now to FIGS. 1A and 1B, there are shown two states of operation of the diffuser device 10. The operational state is shown in FIG. 1A. In this operational state, it is seen that the device 10 includes a main housing 12 which holds a majority of internal functional components, (which are described in detail hereinbelow with reference to FIG. 3), and a cover 14. In the operational state of FIG. 1A, a nozzle 18 is exposed near the upper edge of the housing 12 because the cover 14 is located at a lowermost location in the orientation shown. An atomized form of a disinfecting fluid F is emitted from the nozzle 18. A display screen 16 located on the cover 14 displays the “ON/OFF” status, the battery charge level and the level of fluid in a reservoir which will be described in more detail below. In alternative embodiments, additional or different information about status states of the device 10 and/or the environment may be displayed including data derived from additional sensors in the housing 12, or obtained from the internet via a cellular modem (not shown) provided in the housing 12. Examples of such data may include, but are not limited to: time of operation provided by a timer, temperature, relative humidity, fine particles (pm2.5), air quality index, temperature, and information regarding pathogens known to be in the area.

In FIG. 1B, it is seen that the cover 14 is shifted upward relative to its position in FIG. 1A such that an upper portion of the cover 14 is disposed above the nozzle 18. The cover 14 may be configured with a set of inner grooves or channels (not shown) to slide along a set of ridges or rails (not shown) formed in an upper surface of the housing 12. In this particular embodiment, the cover 14 has functionality as an on/off switch, which will be described in more detail hereinbelow. Therefore, FIG. 1B indicates the shut-off state of the device 10. The screen 16 may be coupled to a microcontroller (not shown) having its own power source in order to operate the screen 16 while the device is in the shut-off state.

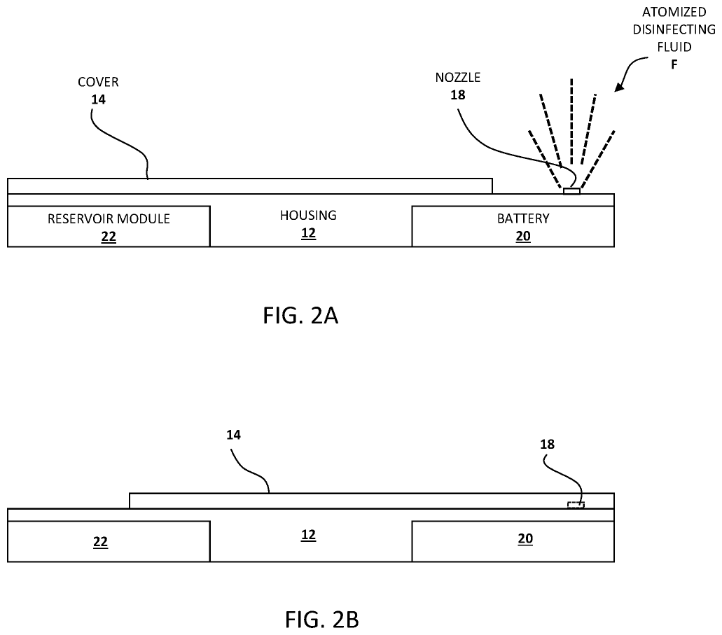

Turning now to FIGS. 2A and 2B, there are shown side elevation views of the device in the operational state (FIG. 2A) and in the shut-off state (FIG. 2B). It is seen in FIGS. 2A and 2B that the device includes a battery 20 and a reservoir module 22. The battery 20 provides electrical power to the device and the reservoir module 22 holds the disinfecting fluid F. Both modules 20 and 22 are removable from the housing 20 to facilitate replacement or charging of the battery 20 and refilling of the reservoir 22 with disinfecting fluid F.

FIG. 3 schematically illustrates additional components of the device 10 with the cover 14 shifted away from its movable position on the top face of the housing 12 in order to clarify visualization of components contained within the housing 12 and the reservoir module 22. In this embodiment, the battery module 20 does not include any components which are not associated with battery functions. Therefore, it is to be understood that conduit 38 extending from the reservoir 24, the nozzle 18, the membrane 36 and electrical conduits 40a, 40b, and 42 all reside within a portion of the housing 12 above the battery module 20. In this particular embodiment, the battery is a 5 volt battery to provide sufficient electrical power for the functionality of the device 10. Alternative embodiments may use a battery providing higher voltage if additional optional components are included and require additional voltage.

The reservoir module 22 includes a reservoir 24 for holding the disinfecting fluid F. The reservoir 24 is surrounded by a jacket 26 which is included to provide temperature control to the reservoir 24, thereby retaining the disinfecting fluid F at a suitable temperature for retaining its disinfecting functionality and for deploying the disinfecting fluid into the conduit to the membrane 36 and nozzle 18. The reservoir module 22 is provided with an upper fill port 28 having a slidable cover 30 connected thereto. The temperature control is useful to retain the fluid in a state suitable for deployment. This is a concern particularly in colder climates where personal devices may be inadvertently subjected to freezing temperatures while being stored in a vehicle, for example. The temperature control functionality may be configured to run continuously from the battery 20, even when the device 10 is shut off, provided another power source, such as a smaller replaceable battery is provided (not shown). In some embodiments, the continuous temperature control may be disabled to conserve battery power if temperature control is not required in a moderate climate or season.

The housing 12 also contains an ultrasonic emitting device 34 which is configured to impart ultrasonic waves to the disinfecting fluid F in the reservoir. The ultrasonic waves generated within the disinfecting fluid causes the fluid to move through the conduit 38 and through the membrane to be emitted from the device via the nozzle 18. In some embodiments, the ultrasonic emitter emits ultrasound waves at frequencies between about 1.6 mHz to about 1.8 mHz. In this particular embodiment, the ultrasonic emitting device 34 is placed in close contact with the conduit 38 for communication of the ultrasonic waves to the disinfecting fluid F in the conduit 38. In alternative embodiments, the ultrasonic emitting device 34 may be placed in an alternative location, such as within the reservoir 24. The ultrasonic emitting device 34 is configured to provide sufficient ultrasonic wave intensity to force the disinfecting fluid F from a reservoir 22 of a volume between about 7 to about 15 mL through the conduit 38 and through the membrane 36, while being powered by a 5 volt battery. Alternative embodiments may have a larger reservoir, a larger conduit and require a battery providing higher voltage.

The main power wiring is indicated by lines 40a-g and it is seen that electrical power is provided to power the jacket 26, the screen 16 and the ultrasonic emitter 34. Signal wiring indicated by lines 42, 44 and 46 extends from the screen 16 to the membrane 36, the ultrasonic emitter 34 and the jacket 26, respectively.

The housing 12 also holds a switch 32 which is activated by sliding the cover 14 downward in the orientation shown.

In some embodiments, the membrane is a chitosan nanofiber membrane, such as the membrane described in US Patent Application No. 20160367722, incorporated herein by reference in its entirety. In some embodiments, the nanofiber membranes have openings with a mean diameter in the range of about 500 nm to about 2.2 μm. These and other alternative membranes are configured in association with the nozzle 18 to cause emission of an atomized stream of particles including nano-filaments of a disinfecting polymer, such as chitosan, in the form of a mist or fine spray.

In some embodiments, the disinfecting fluid used in the device includes a chitosan derivative. In some embodiments, the chitosan derivative is cross-linked. In some embodiments, the chitosan derivative includes and O-sulfonyl modification at the 3-position of the D-glucosamine and/or the N-acetyl-D-glucosamine units.

In some embodiments, the nozzle is provided with the capability to generate an electrostatic atomization. The inner cavity of the nozzle includes a thin film made of positively changed chitosan fibers. When disinfecting liquid F which includes negative ions passes through the nozzle, an electric field is formed, thereby providing the effect of an electrostatic nozzle. By imparting positive charges to the disinfecting polymer enhanced disinfecting activity is provided. In other embodiments, an electrostatic spray is generated according to the principles described in US Patent Application No. 20070194157, incorporated herein by reference in its entirety.

Another aspect of the technology is a kit which includes an embodiment of the disinfecting device as described herein and instructions for operating the disinfecting device. In some embodiments, the instructions include operational details for turning the device on and off by sliding the cover and for removing the battery module and the reservoir module. In some embodiments, the instructions include details regarding filling the reservoir. Some embodiments of the kit include a container of disinfecting fluid, which may be a chitosan solution as described herein. The chitosan solution may include a derivative of chitosan including cross-linkages and/or at least one chemical modification. The chemical modification may be a C-3 O-sulfonyl modification. The kit may also include a battery charger compatible with the battery module 20.

### EQUIVALENTS AND SCOPE

Other than described herein, or unless otherwise expressly specified, all of the numerical ranges, amounts, values and percentages, such as those for amounts of materials, elemental contents, times and current rate, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Unless otherwise defined, all technical and scientific terms used herein have the same meaning as commonly understood by one of ordinary skill in the art.

While the technology been particularly shown and described with references to embodiments thereof, it will be understood by those skilled in the art that various changes in form and details may be made therein without departing from the scope of the technology encompassed by the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

It is also noted that the term “comprising” is intended to be open and permits but does not require the inclusion of additional elements or steps. When the term “comprising” is used herein, the term “consisting of” is thus also encompassed and disclosed. Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the technology, to the tenth of the unit of the lower limit of the range, unless the context clearly dictates otherwise. Where the term “about” is used, it is understood to reflect +/−10% of the recited value. In addition, it is to be understood that any particular embodiment of the present technology that falls within the prior art may be explicitly excluded from any one or more of the claims. Since such embodiments are deemed to be known to one of ordinary skill in the art, they may be excluded even if the exclusion is not set forth explicitly herein.

What is claimed is:

1. A disinfecting device comprising:
a main housing;
a battery module configured for installation within a first receptacle in the main housing;
a reservoir module configured for installation within a second receptacle in the main housing, the reservoir configured to hold a disinfecting fluid;
an ultrasonic wave emitter mounted in the housing and in electrical power communication with the battery, the ultrasonic wave emitter providing ultrasonic waves to the disinfecting fluid thereby creating a pressurized fluid;
a membrane in contact with the pressurized fluid, the membrane for generating reduced-size particles of the pressurized fluid; and
a nozzle mounted on the main housing, the nozzle receiving and emitting a stream of the reduced-size particles of disinfecting fluid.

2. The disinfecting device of claim 1, wherein the reservoir module further comprises a temperature controller in electrical power communication with the battery.

3. The disinfecting device of claim 1, further comprising a user interface screen configured to display an indicator selected from the group consisting of: power status, battery level and reservoir fluid level.

4. The disinfecting device of claim 1, wherein the housing includes a conduit extending from the reservoir to the membrane and wherein the ultrasonic wave emitter is configured to emit ultrasonic waves to the disinfecting fluid in the conduit.

5. The disinfecting device of claim 1, wherein the nozzle is configured to generate the reduced-size particles in an electrostatic spray.

6. The disinfecting device of claim 5, wherein the nozzle has an inner cavity coated with a film of chitosan fibers.

7. The disinfecting device of claim 6, wherein the chitosan fibers are negatively charged.

8. The disinfecting device of any one of claim 1, wherein the membrane is a chitosan nanofiber membrane.

9. The disinfecting device of claim 8, wherein the chitosan nanofiber membrane has openings with a mean diameter in a range of about 500 nm to about 2.2 μm.

10. The disinfecting device of claim 1, wherein the reservoir module is removable and includes a port for refilling the reservoir.

11. The disinfecting device of claim 1, wherein the reservoir module is configured to hold a volume of about 7 mL to about 15 m.

12. The disinfecting device of claim 1, wherein operation of the device is controlled by a housing cover in slidable contact with an upper surface of the housing, the housing cover inactivating the electrical power in a first position on the housing which covers the nozzle, and activating the electrical power in a second position on the housing which exposes the nozzle.

13. The disinfecting device of claim 12, wherein the screen is mounted on the housing cover.

14. The disinfecting device of claim 1, wherein the ultrasonic wave emitter generates ultrasonic waves in a range of about 1.6 mHz to about 1.8 mHz.

15. A disinfecting kit comprising:
the disinfecting device as recited in any one of claims 1 to 14; and instructions for operating the disinfecting device.

16. The kit of claim 15, further comprising a disinfecting fluid.

17. The kit of claim 16, wherein the disinfecting fluid is a chitosan solution.

18. The kit of claim 17, wherein the chitosan solution comprises a derivative of chitosan including cross-linkages and/or at least one chemical modification.

19. The kit of claim 18, wherein the chemical modification is a C-3 O-sulfonyl modification.

20. The kit of claim 15, further comprising a battery charger compatible with the battery module.
